(19) United States
(12) Patent Application Publication
(10) Pub. No.: US 2015/0094376 A1
(43) Pub. Date: Apr. 2, 2015

(54) Hexamethonium Reverses the Lethal Cardiopulmonary Damages in a Rat Model of Brain Stem Lesions Mimicking Fatal Enterovirus 71 Encephalitis

(71) Applicant: Kaohsiung Veterans General Hospital, Kaohsiung City (TW)

(72) Inventors: Wen-Hsien Lu, Kaohsiung City (TW); Kai-Sheng Hsieh, Kaohsiung City (TW); Ching-Jiunn Tseng, Kaohsiung City (TW)

(21) Appl. No.: 14/504,050

(22) Filed: Oct. 1, 2014

(30) Foreign Application Priority Data
Oct. 1, 2013 (TW) 102135569

(51) Int. Cl. A61K 31/14

(52) U.S. Cl. CPC A61K 31/14
USPC 514/642

(57) ABSTRACT

Disclosed is a method for inhibiting the excessive release of catecholamines of mammals infected by the enterovirus 71 (EV71), and more specially, a method for reversing the cardiopulmonary damages caused from EV71 infection in an animal model by using hexamethoniums. An early administration of a suitable amount of hexamethonium to the rats mimicking enterovirus 71 infection will attenuate the acute excessive release of catecholamines in the body of each rat. Thus, cardiac dysfunction and pulmonary edema generally caused by EV71 encephalitis is prevented and the survival rate of the rats increases.

### FIELD OF THE INVENTION

The present invention generally relates to a method for inhibiting the excessive release of catecholamines of mammals infected by the enterovirus 71 (EV71), and more specially, to a method for reversing the cardiopulmonary damages caused from EV71 infection in an animal model by using hexamethoniums.

### BACKGROUND OF THE INVENTION

Hexamethoniums are ganglionic blockers and generally used to treat mammals' chronic hypertension, peripheral nerves systems disorders and so on. The hexamethoniums also may be used as a growth promoter for mammals as disclosed in, for example, the U.S. Pat. No. 3,397,990 entitled “HEXAMETHONIUM SALTS AS GROWTH PROMOTERS IN ANIMAL FEED COMPOSITIONS”, which was issued to A. Hochstein in Aug. 20, 1968, and therefore is included herein for reference. However, the use of hexmethoniums to treat enterovirus in mammals has not been found in any study or report till now.

Among enterovirus 71 (EV 71) infections, brain stem encephalitis that progresses abruptly to cardiac dysfunction and pulmonary edema causes rapid death within several hours. Currently, there is not any known early indicator or treatment used for monitoring or preventing an unexpectedly fulminant course.

Enteroviral encephalitis generally has a favorable prognosis, except when the cause is enterovirus 71. EV 71 infections have been associated with a variety of clinical presentations, and most patients recover within four to six days. However, EV 71 sometimes causes severe neurologic complications, such as acute rhombencephalitis, mainly in children. Rhombencephalitis, i.e. brain stem encephalitis, is the chief neurological complication and has been present in all of the reported fatal cases.

MRIs of fatal EV 71 patients revealed severely destructive lesions in the area of the nucleus tractus solitarii (NTS). The patients who died suffered short, febrile illnesses, decompensated from respiratory distress, and died rapidly with pulmonary edema within several hours of presentation.

Postmortem studies of EV 71 infection have revealed extensive damage to the medulla and pons with necrosis. Immunofluorescence staining for EV 71 antigens has been positive in the neuronal cytoplasm. However, immunofluorescence staining of heart and lung tissue for EV 71 antigents has been negative. Coagulative myocytolysis and myofibrillar degeneration consistent with catecholamine cardiomyopathy have been found in ventricular specimens, and the lungs have exhibited severe congestive hemorrhage.

Extreme sympathetic storm, including systemic hypertension, high levels of plasma catecholamines, and profound peripheral vasoconstriction, leading to cardiac dysfunction and neurogenic pulmonary edema (NPE) has been described in fatal EV 71 infections.

Clinically, chest x-ray has not demonstrated cardiomegaly and has been similar to those in patients with neurogenic cardiac damage. Neurogenic cardiac damage and NPE of the fatal EV 71 encephalitis are considered to be destructive to the area of the NTS.

Connexin 43 is the most important gap junction protein for cell-to-cell coupling in the normal contractile function between adjacent cardiomyocytes and for endothelial cell communications in lung capillaries. In the heart, connexin 43-formed channels are involved in ischemia/reperfusion injury, and the blockade of a large portion of these can attenuate ischemic myocardial contracture. In acute lung injury, connexin 43-mediated gap junctions spread pro-inflammatory signals in the lung capillary bed. In fulminant EV 71 infections, brain stem encephalitis that progresses abruptly to hypersympathetic storm, cardiac dysfunction and NPE is indicative of poor prognosis. Moreover, the current treatment of NPE is primarily supportive. Early indicators, screening tools, and effective measures to prevent NPE are in short supply in fulminant EV 71 infections.

### SUMMARY OF THE INVENTION

The present inventor, through a long-teen study, finally founded that early administration of ganglionic blockers to the rats mimicking EV 71 infections will attenuate acute excessive release of catecholamines in the rats so as to prevent cardiac dysfunction and pulmonary edema generally caused by the EV 71 encephalitis and to increase the survival rate of the rats infected with EV 71.

Therefore, one objective of the present invention is to provide a method for inhibiting the excessive release of catecholamines of mammals infected with EV 71 by using the ganglionic blockers.

Another objective of the invention is to provide a method for treating the cardiopulmonary damages of mammals infected with EV 71 by using hexamethonium.

Another objective of the invention is to reverse the cardiopulmonary damages in mammal models mimicking EV 71 encephalitis.

### BRIEF DESCRIPTION OF DRAWINGS

FIG. 1A is BP and HR recordings each of the rats induced with 6-OHDA lesions (6-OHDA).

FIG. 1B is BP and HR recording each of the rats with 6-OHDA lesions in which the rats were treated with hexamethonium after the lesions induced (6-OHDA+H).

FIGS. 1C to 1E are time chart showing the MBPs of the rats with 6-OHDA lesions (6-OHDA) vs. rats treated with hexamethonium (6-OHDA+H).

FIG. 2A shows the photographs each of the hearts of the rats induced by 6-OHDA lesions (6-OHDA) and the rats treated with hexamethonium (6-OHDA+H).

FIG. 2B shows the photographs of the cross section each of the hearts of the rats in FIG. 2A at the papillary muscle.

FIGS. 2C and 2D respectively show the photographs of short-axis view of the rat's hearts and the echocardiographic of the M-model of rats.

FIGS. 2E to 2H show the time charts each of EF, IVSd and LVPWd of the rats with 6-OHDA lesions (6-OHDA) vs. the rats treated with hexamethonium (6-OHDA+H).

FIG. 3A shows photographs of representative examples of the hematoxylin and eosin stain of rats' hearts in longitudinal sections.

FIG. 3B shows photographs of rats' hearts in immunohistochemical and immunofluorescence analysis of cardiac troponin T.

FIG. 3C shows photographs of rats' hearts in in-situ qualitative analysis by immunohistochemical staining of brain natriuretic peptide.

FIG. 3D shows photographs of the in-situ qualitative analysis by immunohistochemical staining of connexin 43 gap junction protein.

FIGS. 3E to 3G shows time charts of troponin T, brain natriuretic peptide and connexin 43 each of rats with 6-OHDA lesions (6-OHDA) vs. rats treated by hexamethonium (6-OHDA+H).

FIG. 4A shows photographs each of gross specimens of lungs of rats with 6-OHDA lesions (6-OHDA) vs. rats treated with hexamethonium (6-OHDA+H).

FIG. 4B shows the bar chart of wet-dry lung weight ratio of lungs of rats in FIG. 4A.

FIG. 4C shows the bar chart of total lung weight to body weight ratio of rats in FIG. 4A.

FIGS. 5A to 5C show photographs each of histological changes in lung tissue sections of rats with 6-OHDA lesions (6-OHDA) vs. rats treated with hexamethonium (6-OHDA+H).

FIG. 5D shows time chart of quantitative analysis of in-situ PMN of rats induced with 6-OHDA lesions (6-OHDA) vs. rats treated with hexamethonium (6-OHDA+H).

FIG. 5E shows time chart of quantitative analysis of connexin 43-expressing cells of rats with 6-OHDA lesions (6-OHDA) vs. rats treated with hexamethonium after the lesions (6-OHDA+H).

FIG. 6 is a schematic view showing a possible mechanism of fatal enterovirus 71 encephalitis.

### DETAILED DESCRIPTION OF THE INVENTION

We found that early administration of suitable amount of ganglionic blockers (for example, hexamethonium commonly used in clinical) to the rats having a brain lesion model mimicking lethal EV 71 infections in order to treat the lethal cardiopulmonary damages has reversed the lethal cardiopulmonary damages of the rats.

Because the rats induced with the pulmonary damages will die within 7 hours after the infection, the administration time was chosen at 10 minutes after the brain stem lesion. The amount of hexamethonium administered was about 25 mg per 1 kg of the rat (mg/kg).

It should be noted that though we used Sprague-Dawley rats (Rodentia order) as the animal model in this invention, those skilled in the will understand that other suitable mammals may be used to establish the animal model. The suitable animal includes but not limited to, for example, mammals of ardiodactyla such as domestic pig and mammals of primatology such as chimpanzee and human beings.

### Materials and Methods

### Introduction

Though it is not desired to be limited by any theory, we proposed in this invention that EV 71 induces catecholamine over-expression and results in fatal cardiomyopathy and pulmonary hemorrhagic edema. In this invention, one goal was to use a rat model mimicking fatal EV 71 encephalitis to understand the alterations in cardiopulmonary damage with drug intervention for clinical applications. Thus, we had three specific aims: 1) to establish a rat model of brain stem lesions that would mimic acute hypertension cardiac dysfunction, pulmonary edema and death within hours caused by fatal EV 71 encephalitis; 2) to investigate the time-course and tissue basis of changes in the heart and lungs of rats with NTS lesions; and 3) to evaluate drug treatment through ganglionic blockade to prevent neurogenic cardiopulmonary damage and increase survival time.

### Animal and Experiment Designs

Male Sprague Dawley rats weighted about 270-360 grams (available from the National Science Council Animal Facility) were housed in the animal room of Kaohsiung Veterans General Hospital (Kaohsiung, Taiwan).

Three sets of experiments were designed according to the three specific aims mentioned above. The first set of experiments was to establish a rat model by micro-injecting 6-hydroxydopamin (6-OHDA) into NTS leading to acute fulminating hypertension and pulmonary hemorrhagic edema. We recorded the blood pressure (BP) and heart rate (HR) in trend. Two groups of rats (n=6 in each group) were studied: 1) an intervention group in which 6-OHDA (Sigma, 30 g) dissolved in vehicle (0.8% ascorbic acid in saline) was injected into NTS bilaterally; and 2) a control group that received injections of equal volumes of vehicle alone. Each group of rats was anesthetized with urethane (1 g/kg, i.p.) and connected to oscillator circuits for BP and HR recording via femoral vein. For microinjections into the NTS, the rats were anesthetized and placed in a stereotaxic frame; then the dorsal surface of the medulla was exposed. A glass cannula was filled with L-glutamate (0.154 mmol/60 n1) to functionally identify the NTS. A specific decrease in BP and HR (>1=−35 mmHg and −50 bpm) was demonstrated after microinjection of L-glutamate in the NTS.

After the first set of the rat model was established, the second set of experiments intended to determine some time points for analysis. Rats were anesthetized with pentobarbital (50 mg/kg, ip) before NTS lesion. After surgery (lesion of the NTS), the wounds were closed and the rats were removed from the stereotaxic frame for further observation. Each rat was placed in a small cage and woken within 30 minutes for free activity. The rat were respectively sacrificed (intervention and control group, n=6 in each group) at different time point (0, 3 and 6 hours) for analyzing histopathology and biochemistry from blood, brain, heart and lung. Before sacrificing, echocardiography was performed to exanimate left ventricular (LV) wall thickness and cardiac function.

The third set of experiments (intervention-treatment and control-treatment groups) was to infuse the ganglionic blocker (hexamethonium (Sigma), 25 mg/kg, iv) via the femoral vein at the time point of 10 minutes after NTS lesion to record BP and HR in trend (n=6 in each group) and the rats respectively were sacrificed at the time point (0, 3, 6 and 12 hours) for analyzing the parameters as discussed in the second set of experiments.

The abdomen of the rats was opened for blood sampling from the inferior vena cava. All blood samples were collected into micro-tubes and placed immediately into to an ice bath. The samples, then, were centrifuged at 10000 rpm for 20 minutes at 4° C. Aliquots of plasma were removed and stored at −80° C. until assayed for content of epinephrine and norepinephrine within four weeks utilizing enzyme immunoassay for the high-sensitive quantitative determination by Labor Diagnostika Nord-Medicrop Inc.

### Pulmonary Edema Index and Histopathology

After the blood-sampled rats were sacrificed, the chest each of the rats was opened to remove the lung and the heart, and then the skull was opened for NTS tissue. To estimate the liquid accumulation in the lungs, both lungs were weighted, then dried in an oven at 80° C. for two days and weighted as dry lung weight. The total weight/body multiplies by 100 and wet-to-dry lung weight ratio were calculated for indicating the degree of pulmonary edema.

For hematoxylin and eosin staining, the rat brain, heart, and lungs were excised immediately after blood sampling. They were rinsed with saline, free from blood or fatty tissue, lightly blotted with filter paper, and then were placed in 10% formalin for five days and blocked, embedded in paraffin, cut in 4 μm sections, and stained with hematoxylin and eosin.

Immunohistochemical analysis (troponin T and brain natriuretic peptide in heart tissue, neutrophil cell in lung tissue, and connexin 43 assays in the lung and heart tissues) was performed on the sections after the paraffin was melted in an oven at 70° C. for one hour. The sections were deparaffinized, microwaved in citric buffer (10 mmol/L, pH 6.0), blocked in 3% goat serum, and incubated with anti-cardiac troponin T antibody (1:200; Abeam), anti-myosin VIIa antibody (1:200; Abeam), anti-brain natriuretic peptide polyclonal antibody (1:1000; Thermo Fisher Scientific Inc.), anti-neutrophil elastase antibody (1:400; Abeam), and anti-connexin 43/GJA1 antibody (1:200; Abcam) overnight at 4° C. Next, the sections were incubated with biotinylated secondary antibodies (1:200; Vector Laboratories, Burlingame, Calif., USA) for one hour and in AB complex (1:100) for 30 minutes at room temperature. The sections were visualized with a DAB substrate kit (Vector Laboratories) and counterstained with hematoxylin. The sections were then photographed with a microscope mounted with a charge-coupled device camera.

### Echocardiography

Rats were immobilized and shaved on the chest for echocardiographic examinations using commercially available equipment (Vivid 7, GE Ultrasound, Horten, Norway) equipped with 10-MHz phased array transducers. All the records were stored digitally as two-dimensional cine loops and analyzed with customized dedicated research software (Echo PAC, GE Ultrasound, Horten, Norway). Two-dimensionally guided M-mode recordings were obtained from the parasternal short-axis view at a depth of 2 cm at the level of the papillary muscles. M-Mode tracing were recorded at a speed of 200 mm/s. The measurements were made using the method of the American Society of Echocardiography and the parameters were measured digitally on the M-mode tracing and averaged from 5 consecutive cardiac cycles.

### Statistical Analysis

Group data were expressed as mean+/−SEM. In the same group, the mean differences of BP (or HR) in various time points were evaluated by one-way repeated measures ANOVA and Bonferroni post hoc test. The mean differences of parameters among three intervention/control group (0, 3, and 6 hours) in set 2 of the experiments and among four intervention-treatment/control-treatment groups (0, 3, 6, and 12 hours) in set 3 of the experiments were evaluated by one-way ANOVA with Tukey post hoc test. In addition, Student's t-test was applied to compare two groups (the intervention group in set 2 vs. the intervention-treatment group in set 3) at the same sacrificed time point. P<0.05 was considered statistically significant.

### Results

### Example 1

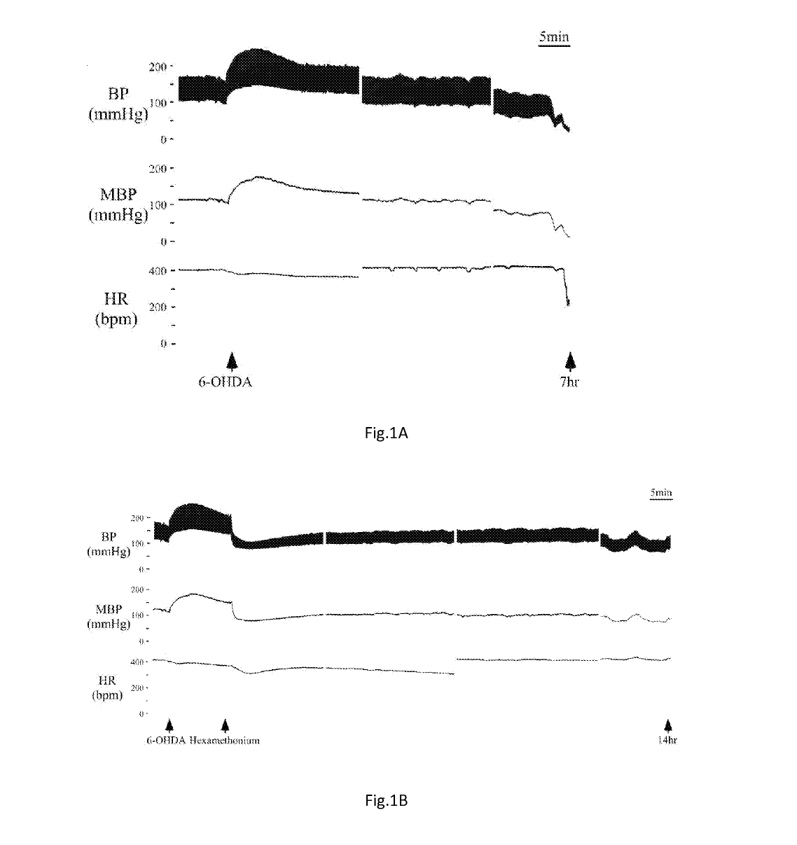

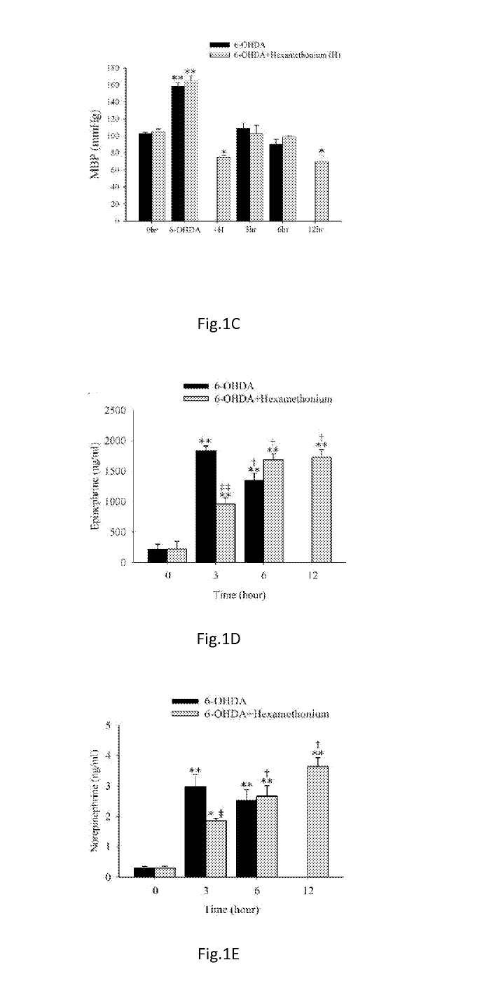

Referring to FIGS. 1A to 1E, a nature history and the changes of catecholamines in hexamethonium treatment vs. placebo were shown. As shown in FIG. 1A, acute hypertension was produced after the rat was administered with 6-OHDA lesions and the rat died suddenly within 7 hours (n=6). As shown in FIG. 1B, the acute hypertension was reversed after hexamethonium administration. All rats survived for at least 14 hours (n=6). As shown in FIG. 1C, a significant acute high rise in mean blood pressure (MBP) caused by 6-OHDA lesions was significantly reversed with hexamethonium treatment (vs. respective 0 hr, n=6 per group). In FIGS. 1D and 1E, similarly, the acute high rise of epinephrine and norepinephrine serum levels post 6-OHDA lesions was significantly lowered within 3 hours after hexamethonium treatment (n=6 per group). Data represent means±S.E.M.*, P<0.05 [symbol] **, P<0.001 vs. respective 0 hr. +, P<0.001 vs. respective 3 hr. ‡, P<0.05 and ‡‡, P<0.001 6-OHDA vs. 6-OHDA+Hexamethonium.

In this example, all rats with bilateral 6-OHDA lesions of the NTS died within 7 hours. The rats exhibited labored breathing with grunting respiration and inspiratory retractions of the suprasternal notch and subcostal area beginning 2 to 3 hours after the NTS lesions were created; these symptoms progressively worsened until death. There was a pink frothy fluid in the rats' nostrils before death. However, the rats that received 6-OHDA+Hexamethonium all survived for more than 14 hours and did not exhibit significantly labored breathing until 14 hours post NTS lesion.

In the BP and HR recording of the rats with 6-OHDA lesions (see FIG. 1A and FIG. 1C), the systolic blood pressure reached a hypertensive plateau within 5 minutes after the NTS was bilaterally microinjected with 6-OHDA, in association with a smaller drift in the diastolic blood pressure and a narrowing followed by a widening of pulse pressure. During this acute hypertensive period, there was no significant change in the HR. Over the next 2 to 3 hours, the blood pressure declined gradually to the baseline levels, and the rats died rapidly within 7 hours, with an acute drop in BP prior to death. In the rats with 6-OHDA lesions that were given hexamethonium 10 minutes after (FIGS. 1B and 1C), acute reversal of the hypertension induced by 6-OHDA occurred immediately, to less than the baseline level with a narrowing of the pulse pressure and a reduction in HR. The BP gradually returned to the baseline level, with normal pulse pressure and no significant reduction in HR at 3 hours. In the control group, there were no significant changes in BP and HR. In the control-treatment group, the mean blood pressure significantly decreased after the administration of hexamethonium, with a narrowing of the pulse pressure and a gradual return to the baseline with no associated significant change in HR. According to rat brain histology based on H-E staining, the rats with NTS bilaterally microinjected with 6-OHDA showed extensive NTS necrosis and bilateral loss of neurons. However, the rats with NTS bilaterally microinjected with vehicle in the control group showed no bilateral NTS necrosis.

In the rats with 6-OHDA lesions, the levels of epinephrine and norepinephrine (FIGS. 1D and 1E) at 3 hours were acutely and excessively higher than baseline levels and remained at high levels until 6 hours. Although there were no significant differences in the levels of epinephrine and norepinephrine at 6 hours between the 6-OHDA and 6-OHDA+Hexamethonium groups, the hexamethonium treatment attenuated the increased levels of epinephrine and norepinephrine at 3 hours.

### Example 2

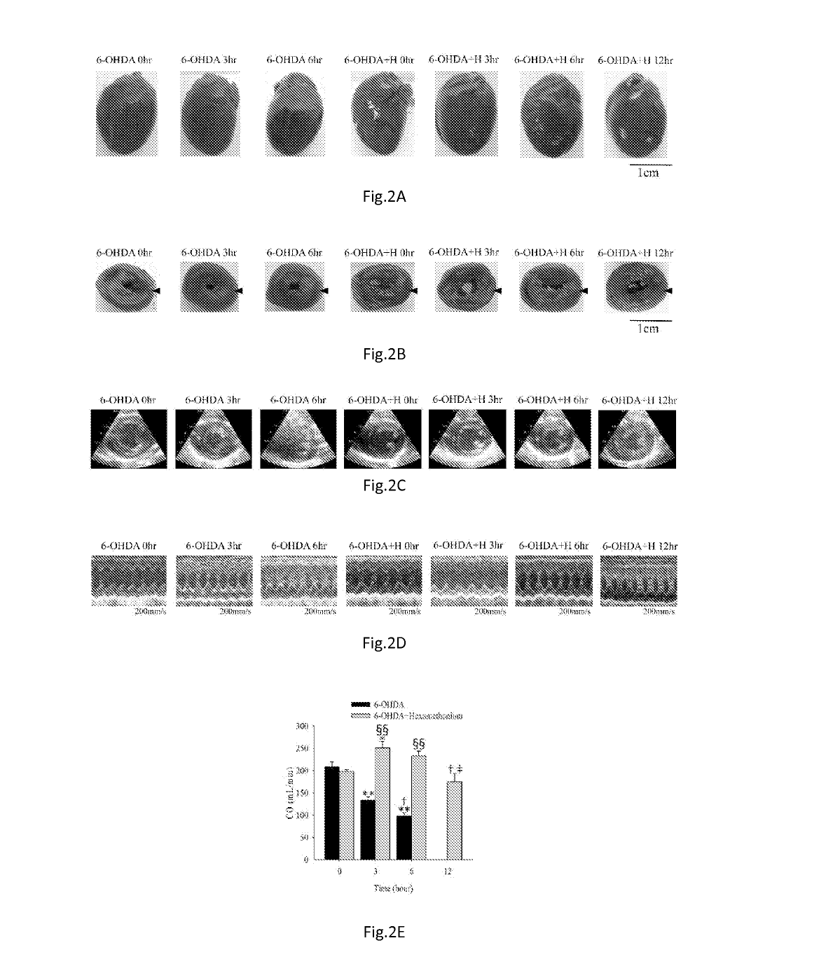

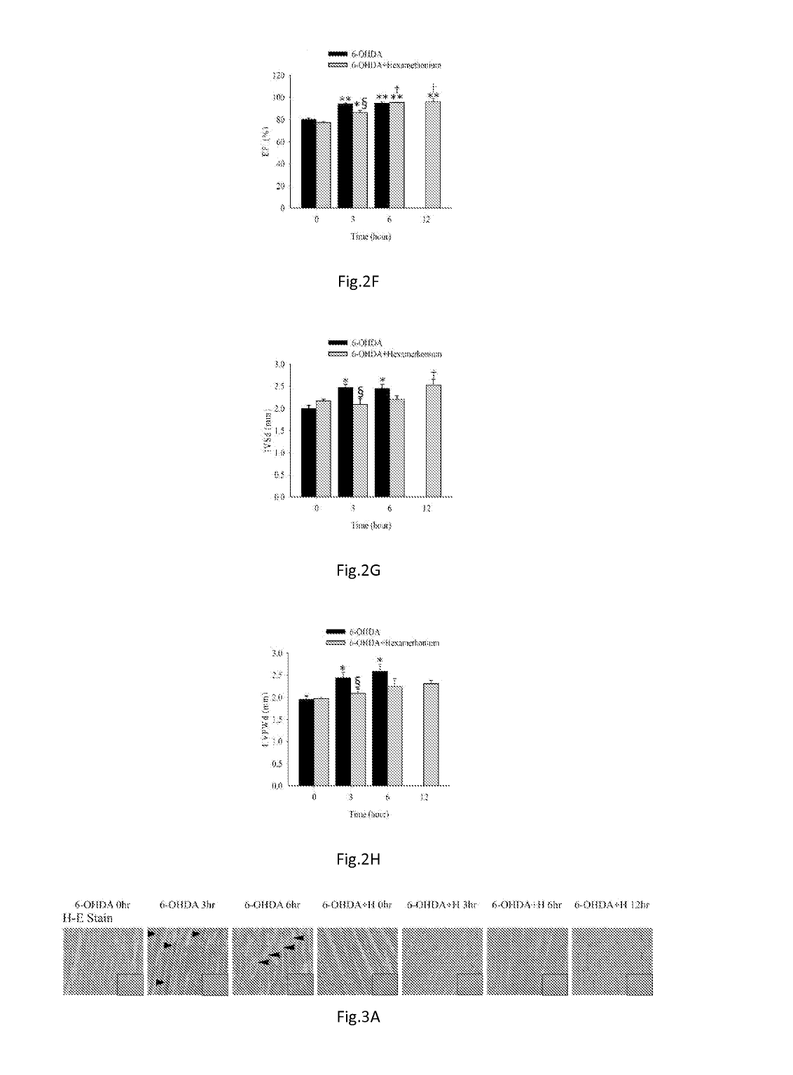

In cardiac morphology, we found that there were no significant changes in the gross specimens of heart (FIG. 2A), but the LV wall thickness increased at 3 hours and 6 hours after 6-OHDA lesion in the cross-section of the heart (FIG. 2B). From echocardiographic 2-dimensional short-axis views (FIG. 2C) and M-model images (FIG. 2D), it was revealed that acutely decrease cardiac output at 3 hours and 6 hours after 6-OHDA lesions were prevented with hexamethonium treatment (FIG. 2E). The acute increase in ejection fraction (FIG. 2F), fraction shortening, and LV wall thickness (FIGS. 2G and 2H) were attenuated or reversed at 3 hours with hexamethonium treatment. In addition, the decrease in the LV internal dimension in diastole and systole were reversed or attenuated at 3 hours and 6 hours with hexamethonium treatment. Data represent means±S.E.M.*, P<0.05 and **, P<0.001 vs. representative 0 hr. +, P<0.05 vs. representative group 3 hr. ‡, P<0.05 vs. representative group 6 hr. §, P<0.05 and §§, P<0.001 6-OHDA vs. 6-OHDA+H.

### Example 3

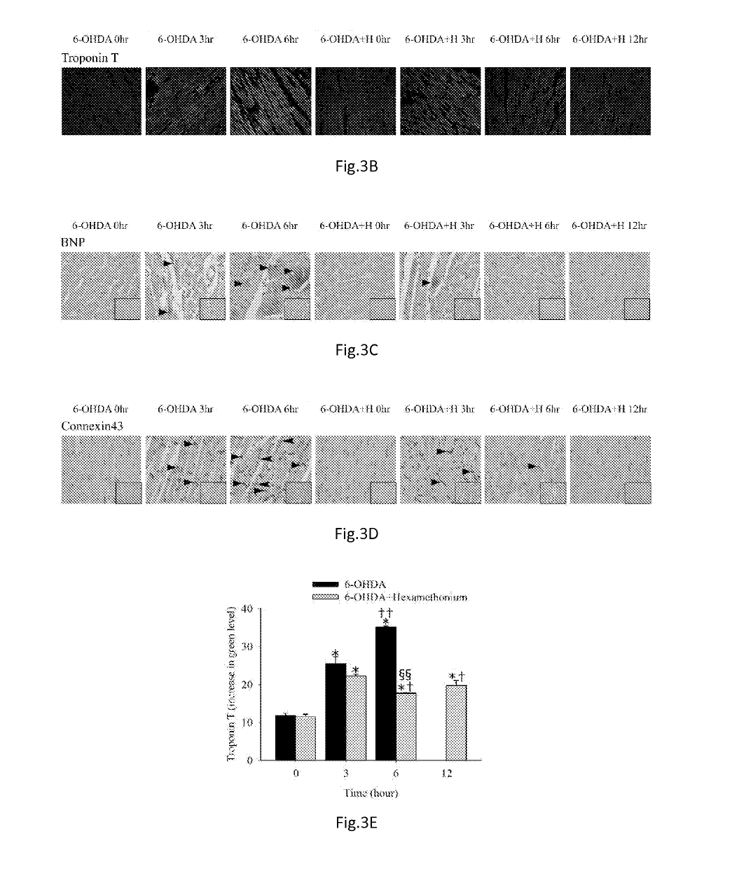

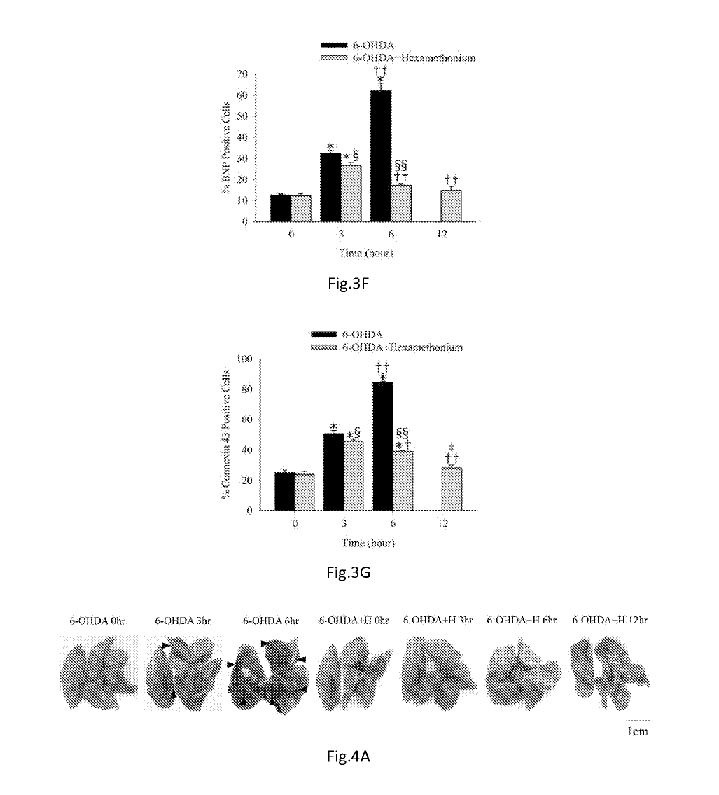

Referring to FIGS. 3A to 3G, pathological changes on hearts specimens in hexamethonium treatment (+H) vs. placebo (original magnificent 400×; inset original magnificent 100×) were shown. FIG. 3A was regarded with representative examples of the hematoxylin and eosin stain. As shown in FIG. 3A, the increase of the irregular wavy fibers with increased eosinophilic staining of the cytoplasm (indicated by the arrowhead) and contraction bands necrosis (indicated by the concave arrowhead) at 3 hours and 6 hours in the 6-OHDA group was prevented by hexamethonium treatment (n=6 per group). FIGS. 3B and 3E showed the representative examples of cardiac troponin T (original magnification 280×). Myocardial section co-stained with antibodies directed against troponin T antibody (green) and nuclei were stained by DAPI (blue). The increased expression of troponin Tat 6 hours in the 6-OHDA group was reduced by hexamethonium treatment (n=4 per group). FIG. 3C showed representative examples of brain natriuretic peptides (BNP). The increased BNP staining in the cytoplasm at 3 hours and 6 hours in the 6-OHDA group was reduced by hexamethonium treatment (n=6 per group). FIG. 3D showed representative examples of immuno-cytochemistry detection of connexin 43 in rat heart muscle sections. As shown, the increased and strong expressions of connexin 43 in intercalated discs (indicated by the arrowhead) and between adjacent myofibers (indicated by the concave arrowhead) at 3 hours and 6 hours in the 6-OHDA group was reduced by hexamethonium treatment (n=6 per group). FIGS. 3F and 3G showed the graphs depicting quantitative analysis of in situ BNP and connexin 43-expressing cells in rat heart. Data represent means±S.E.M.*, P<0.001 vs. respective group 0 hr. +, P<0.05 and ++, P<0.001 vs. respective group 3 hr. ‡, P<0.001 vs. respective group 6 hr. §, P<0.05 and §§, P<0.001 6-OHDA vs. 6-OHDA+Hexamethonium.

Regarding the histological changes of myocardial fibers in longitudinal sections, please refer to FIG. 3A, the appearance of irregular wavy fibers, increased eosinophilic staining of the cytoplasm, and contraction band necrosis reflected the hypercontracted state of the cells at 3 hours and 6 hours in the 6-OHDA group, which was prevented by hexamethonium treatment. In the immunohistochemical and immunofluorescence analysis of cardiac troponin T, please refer to FIG. 3B and the in situ qualitative analysis by immunohistochemical staining of brain natriuretic peptide, please refer to FIG. 3C and connexin 43 gap junction protein, please refer to FIG. 3D, increased and strong expressions of tropnin T, brain natriuretic peptide and connexin 43 caused by 6-OHDA lesions decreased within 3 to 6 hours after hexamethonium treatment, please refer to FIGS. 3E to 3G.

### Example 4

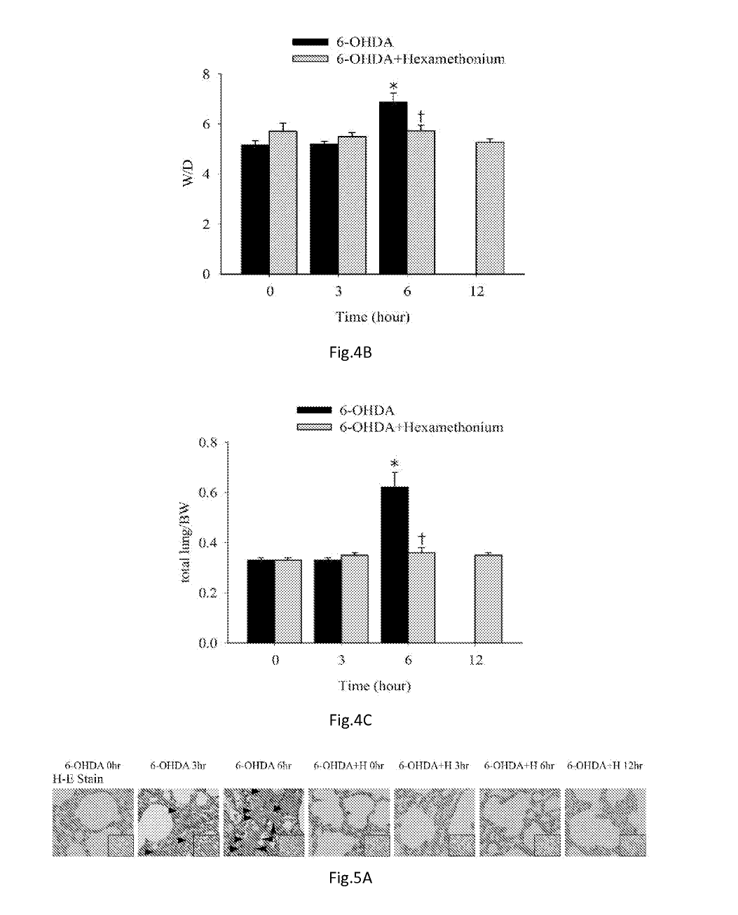

FIGS. 4A to 4C showed the changes of pulmonary hemorrhagic edema in hexamethonium treatment (+H) vs. placebo. Referring to FIG. 4A, representative examples of gross of rat lungs were shown. The mild and sever lung congestion (indicated by arrowhead) were both induced in rats with 6-OHDA lesions at 3 hours and 6 hours and were prevented by hexamethonium treatment (n=6 per group). Referring to FIGS. 4B and 4C, the wet-to-dry lung weight ratio (W/D) and total lung/body weight (BW) were increased at 6 hours in the 6-OHDa group and reversed by hexamethonium treatment (n=6 per group). Data represent ±S.E.M.*, P<0.001 vs. respective group, +P<0.05 6-OHDA vs. 6-OHDA+H.

Based on the gross specimens of the lung, please refer to FIG. 4A, wet-to-dry lung weight ratio, please refer to FIG. 4B, and total lung-to-body weight after NTS lesions, please refer to FIG. 4C, there was mild lung congestion without edema at 3 hours. However, the rats developed severe pulmonary hemorrhagic edema within 6 hours after 6-OHDA lesion. Pulmonary edema caused by 6-OHDA lesion at 6 hours was reversed by hexamethonium treatment.

### Example 5

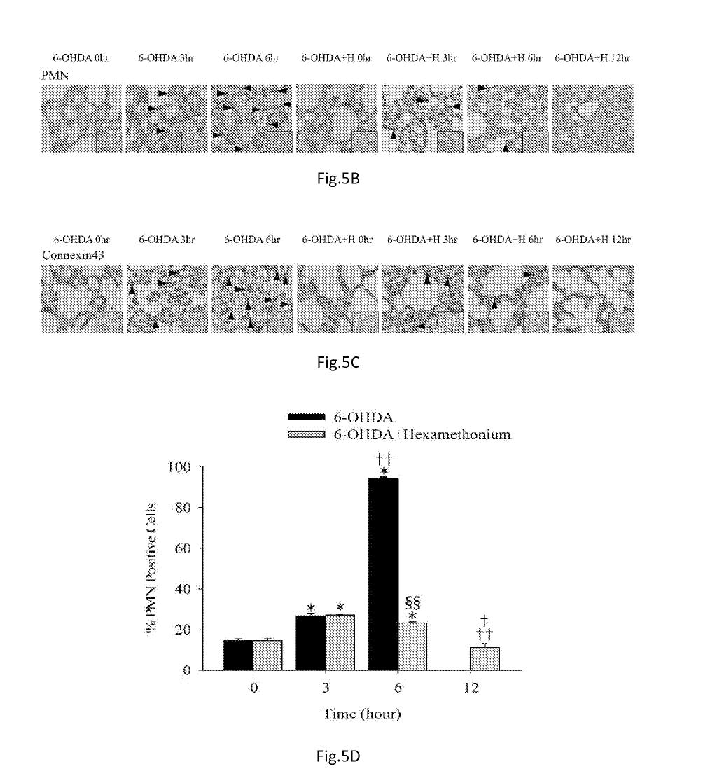

FIGS. 5A to 5E showed pathological changes on lung specimens in hexamethonium treatment (+H) vs. placebo (original magnification 400×; inset original magnification 100×). FIG. 5A showed representative examples of hematoxylin and eosin stain. As shown in FIG. 5A, the increased infiltrates of red blood cellsat 3 hours, and with the formation of hyaline membrane (indicated by arrowhead) at 6 hours in the 6-OHDA group, which were prevented by hexamethonium treatment (n=6 per group). FIG. 5B showed representative examples of polymorphnuclear neutrophil (PMN) (indicated by arrowhead). The increased infiltrates of PMN at 6 hours in the 6-OHDA group were reduced by hexamethonium treatment (n=6 per group). FIG. 5C showed representative examples of connexin 43. The increased expressions of connexin 43 (indicated by arrowhead) with intensely positive at their membrane interfaces at 3 hours and 6 hours in the 6-OHDA group was attenuated by hexamethonium treatment (n=6 per group). FIGS. 5D and 5E were graphs depicting quantitative analysis of in situ PMN and connexin 43-expressing cells. Data represent means±S.E.M.*, P<0.001 vs. respective group 0 hr. +, P<0.05 and ++, P<0.001 vs. respective group 3 hr. ‡, P<0.001 vs. respective 6 hr. §, P<0.05 and §§, P<0.001 6-OHDA vs. 6-OHDA+H.

As to histological changes in lung tissue sections shown in FIG. 5A, the appearances of increased cell infiltration and red blood cells in the lung interstitium worsened, with hyaline membrane formation at 3 hours and 6 hours in the 6-OHDA group, which was attenuated or prevented by hexamethonium treatment. The infiltrating cells were found to be neutrophils by immune-histochemical staining, please refer to FIGS. 5B and 5D. The 6-OHDA-induced increase in connexin 43 decreased time-dependently after hexamethonium treatment, please refer to FIGS. 5C and 5E.

### CONCLUSION

From the examples mentioned above, we found that rats with 6-OHDA-induced NTS lesions experienced acute hypertension and acutely large amounts of epinephrine and norepinephrine released into the serum within 10 minutes after lesion creation (data does not shown). The levels of catecholamine were persistently higher until death. Severe cardiac dysfunction with low CO was found within 3 hours after 6-OHDA lesion, and death from severe pulmonary hemorrhagic edema finally occurred within 7 hours as a result of LV failure. The course of 6-OHDA-induced NTS lesions resembled that of fatal EV 71 encephalitis, which is thought to damage the area of the NTS. Clinically, an EV 71 infection with brain stem encephalitis is a high-risk indicator for abrupt and critical progression to cardiac dysfunction, NPE and death within several hours. In addition, NPE with fulminant EV 71 is thought to be associated with LV failure.

In this rat model with NTS lesion, although the serum levels of epinephrine and norepinephrine at 6 hours in the 6-OHDA+Hexamethonium group were as high as those in the 6-OHDA group, cardiopulmonary damage was prevented (preserving CO and preventing pulmonary hemorrhagic edema). Early hexamethonium treatment reduced the levels of epinephrine and norepinephrine within 3 hours to attenuate the effects of high circulating catecholamines and prevent further injury to the heart and lungs. In other words, acute excessive and persistently high level of catecholamines will cause acute cardiac dysfunction and pulmonary edema. However, lowered levels of catecholamines will prevent further cardiopulmonary damage from subsequent higher levels of catecholamines. This finding was similar to that of a previous study in which an injection of a large dose of catecholamines rapidly and abruptly resulted in severe pulmonary edema and hemorrhage. However, with a slow infusion of catecholamine, the degree of pulmonary changes was milder. Thus, when the sympathetic storm is building up slowly, hemodynamic changes will be not as acute and intense, allowing the cardiovascular system to adjust. This was also reflected in the expression of connexin 43 in our study. The expression of connexin 43 increased in the heart and lungs after 6-OHDA lesions and these increases were reversed with hexamethonium treatment.

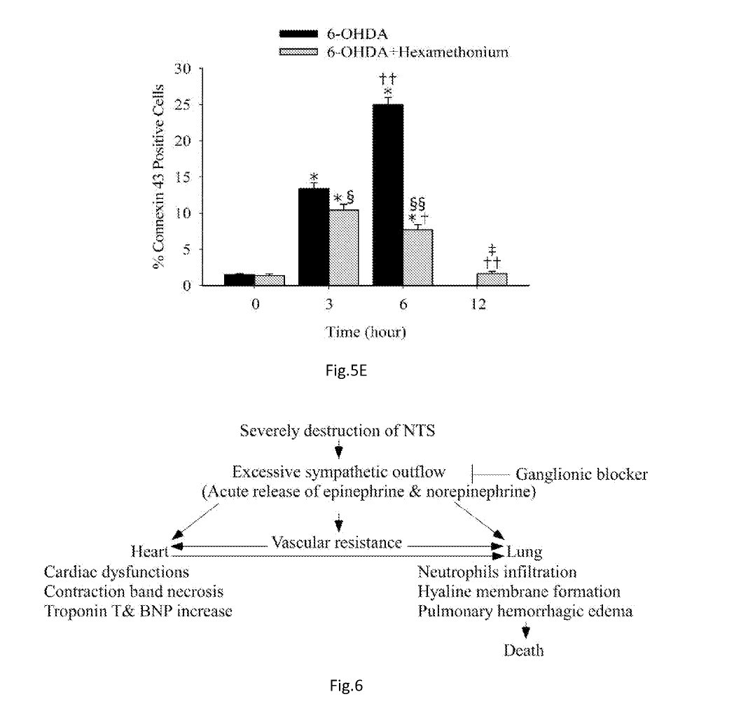

Though not intended to be limited by any theory, we proposed possible mechanism for fatal EV 71 encephalitis, please refer to FIG. 6. The NTS is richly innervated with catecholamine-containing neurons, and the axons of these neurons synthesize and release catecholamines. When EV 71 severely attacks the NTS area, the NTS cannot modulate the activity of the autonomic neurons in the medulla that, in turn, regulate autonomic control. The excessive destruction of the NTS causes marked augmentation of sympathetic nerve activity, and large amount of epinephrine and norepinephrine surge in the blood, resulting in injuries to the resistant blood vessels, heart, and lungs. Sympathetic overactivation and catecholamine toxicity can cause peripheral vasoconstriction, necrosis of myocardial cells, and increased permeability of the pulmonary capillaries, leading to fatal hemorrhagic edema.

In animal models, EV 71 can spread from the gastrointestinal tract to accumulate in the brain stem of rats via retrograde axonal transport or blood flow pathways after inoculation. In the brain stem, EV 71 can bind to the receptors of P-selection glycoprotein ligand-1 and scavenger receptor class B member 2. These two receptors facilitate viral attachment and entry and induce inflammatory cytokines to disrupt the blood-brain barrier, thereby permitting viral invasion. However, understanding why EV 71 has a greater tendency to target NTS neurons than other viruses still requires further study.

Hyertension, cardiac dysfunction and NPE are extremely uncommon as consequences of infectious disease. Although our results could be applied to any disease involving NTS lesions, thus far, the fulminant EV 71 encephalitis in outbreaks in Taiwan (since 1998) and in other area of the world have been consistent with a disease course of NTS destruction. Early recognition of critical cases that will progress to the complications of NPE after EV 71 infection is the most important measure to be undertaken before an EV 71 vaccine is invented in the future.

For translational research of fulminant EV 71 encephalitis, this study demonstrated interactive relationships among brain stem encephalitis, cardiomyopathy, and neurogenic pulmonary edema in a rat model mimicking fatal EV 71 encephalitis. We identified acute hypertension as an early indicator, echocardiography as a useful monitoring tool, and attenuated the effects of high circulating catecholamines by administrating a ganglionic blocker as a preventive measure for cardiac dysfunction and neurogenic pulmonary edema in brain stem lesions.

Referring to FIG. 6, a possible mechanism of fatal enterovirus 71 encephalitis is shown. When nucleus tractus solitarii (NTS) is severely damaged by enterovirus 71, it will excessively increase activity of pre-ganglionic neurons, excessive sympathetic outflow, and acute largely epinephrine/norepinephrine release. The sympathetic overacitivation and catecholamine toxicity will cause peripheral vasoconstriction, severe heart strain and failure, loading in the pulmonary circulation, finally leading to fatal pulmonary hemorrhagic edema. When treated earlier with pre-ganlionic blocker (hexamethonium) after NTS destruction, it will attenuate the rapidly excessive release of epinephrine and norepinephrine to prevent lethal cardiac dysfunction and the production of fatal pulmonary edema.

In conclusions, by administrating hexamethonium in an animal model mimicking fatal EV71 encephalitis can indeed reverse the cardiac dysfunction and reduce the death rate. Therefore, the present invention is an all new invention which has novelty and inventive step.

What is claimed is:

1. A method for inhibiting acute release of catecholamines of mammals infected by the enterovirus 71, comprising:
administrating a suitable amount of a ganglionic blocker to the mammals infected with enterovirus 71 encephalitis.

2. The method for inhibiting acute release of catecholamines of mammals infected by the enterovirus 71 as claimed in claim 1, wherein the ganglionic blocker is hexamethonium.

3. The method for inhibiting acute release of catecholamines of mammals infected by the enterovirus 71 as claimed in claim 2, wherein the amount of administration of hexamethonium is within a range from about 10 mg/kg to about 30 mg/kg.

4. The method for inhibiting acute release of catecholamines of mammals infected by the enterovirus 71 as claimed in claim 2, wherein the amount of administration of hexamethonium is about 25 mg/kg.

5. The method for inhibiting acute release of catecholamines of mammals infected by the enterovirus 71 as claimed in claim 2, wherein the administration of hexamethonium is conducted early after infection of enterovirus 71.

6. The method for inhibiting acute release of catecholamines of mammals infected by the enterovirus 71 as claimed in claim 2, wherein the administration of hexamethonium is conducted within six hours after infection of enterovirus 71.

7. The method for inhibiting acute release of catecholamines of mammals infected by the enterovirus 71 as claimed in claim 1, wherein the mammals comprises mammals in Rodentia order.

8. The method for inhibiting acute release of catecholamines of mammals infected by the enterovirus 71 as claimed in claim 1, wherein the mammals comprises mammals in Euarchonta order.
